(19) United States
(12) Patent Application Publication
(10) Pub. No.: US 2026/0094678 A1
(43) Pub. Date: Apr. 2, 2026

(54) APPARATUS FOR DETERMINING A TECHNICAL APPLICATION PROPERTY OF A SUPERABSORBENT MATERIAL

(71) Applicant: BASF SE, Ludwigshafen am Rhein (DE)

(72) Inventors: Thomas DANIEL, Ludwigshafen am Rhein (DE); Christophe BAUDUIN, Ludwigshafen am Rhein (DE)

(73) Assignee: BASF SE, Ludwigshafen am Rhein (DE)

(21) Appl. No.: 19/113,497

(22) PCT Filed: Sep. 22, 2023
(86) PCT No.: PCT/EP2023/076204

§ 371 Date: Mar. 20, 2025

(30) Foreign Application Priority Data
Sep. 23, 2022 (EP) 22197271.4

(51) Int. Cl. G16C 60/00; G16C 20/30; G16C 20/70

(52) U.S. Cl. CPC G16C 60/00; G16C 20/30; G16C 20/70

(57) ABSTRACT

The disclosure relates to an apparatus and methods for predicting the absorption capacity, swelling kinetics or permeability of a superabsorbent material used in e.g. diapers. The superabsorbent material comprises polymer particles having an interconnected core and a surface cross-linked shell. The absorption capacity, swelling kinetics or permeability of the material are predicted based on the size distribution of the particles using a trained model. The output of the apparatus and method is control data for adjusting a manufacturing process so as to produce particles having a size distribution that provides a desired target absorption capacity, swelling kinetics or permeability.

### FIELD OF THE INVENTION

The invention relates to an apparatus, a method and a computer program product for determining a technical application property of a superabsorbent material. Moreover, the invention refers to an interface apparatus, an interface method and an interface computer program product for providing an interface for determining a technical application property of a superabsorbent material. Furthermore, the invention refers to a training apparatus, training method and training computer program product for parameterizing a property determination model utilizable for determining a technical application property of a superabsorbent material.

### BACKGROUND OF THE INVENTION

In many modern sanitary products, superabsorbent materials, often in form of superabsorbent particles, are utilized for fluid absorption. However, the production process of the necessary superabsorbent particles is complex and although this process comprises a plurality of grinding and classification steps, which are typically sieving steps, the resulting particles still vary in size. The respective equipment in such a powder process is subject to intensive wear and tear which leads to drifting grinding and sieving properties. Moreover, due to the complex production process the continuous adjustment of the process is necessary to provide a product with a continuous product quality, in particular, a product providing always the same technical application properties, in particular, the same absorption characteristics. Currently, the readjusting of the production process to provide a superabsorbent material always with the same technical application properties is mostly done based on experience of a person controlling the production process or by random sampling and determining adjustments based on measurements of the random samples. Furthermore, in the current design process for determining the characteristics of the superabsorbent material to be utilized in, for example, a sanitary product, generally laboratory experiments are utilized and the characteristics of respectively designed particles are measured. However, these measurements often only provide information on the technical application properties of the optimal superabsorbent particle, whereas during the normal production the produced superabsorbent particles will often vary and thus will be distributed around the optimal superabsorbent particle. This can lead to deviations in the technical application property of the produced superabsorbent particles from the predicted technical application properties, wherein these deviations are difficult to predict due to the design process and a readjusting of the production is often not possible or only possible by a redesign of the respective superabsorbent particle. Such a redesign typically includes process and formulation adjustments.

It would thus be advantageous if the technical application properties of the superabsorbent particle could be predicted more easily and accurately enough to allow for a controlling of the production process of superabsorbent particles based on the technical application property prediction.

### SUMMARY OF THE INVENTION

It is an object of the present invention to provide an apparatus, a method and a computer program product that allows for an improved determination of technical application properties of superabsorbent materials that allows for an improved controlling of a production process of a superabsorbent material, in particular, to produce a superabsorbent material that continuously meets a predetermined technical application property. Moreover, it is further an object of the invention to provide a training method, a training apparatus and a computer program product that allow to provide a property determination model that is suitable to be utilized in the method, apparatus and computer program product.

In a first aspect of the invention, an apparatus for determining a technical application property of a superabsorbent material is presented, wherein the superabsorbent material is provided in form of superabsorbent particles comprising a superabsorbent polymer provided in form of i) an interconnected core and ii) a surface cross-linked shell with a higher connectivity than the core, wherein the apparatus comprises a) a receiving interface receiving a particle size of the superabsorbent particles of a superabsorbent material, b) one or more processors configured to utilize a property determination model for determining the technical application property of the superabsorbent material based on the particle size, wherein the property determination model is a data-driven model that has been parameterized such that it is adapted to determine a technical application property of a superabsorbent particle based on the size of the particle, and c) an output interface for generating control data based on the determined technical application property.

It has been found by the inventors that it is possible to train a property determination model that allows to determine a technical application property of a superabsorbent particle in an accurate manner based on the size of the respective superabsorbent particle. Accordingly, technical application properties of superabsorbent materials comprising superabsorbent particles with different sizes, i.e. comprising a size distribution, can be easily predicted by determining the technical application properties for each size present in the superabsorbent material. Thus, by utilizing such a property determination model to determine a technical application property of a superabsorbent particle based on the size of the particle, control data can be generated that allows for an improved controlling of the production of the superabsorbent material, for instance, by adjusting the produced size distribution of the particles forming a superabsorbent material. In particular, the production of a superabsorbent material can be directly controlled based on an easily measurable variable, i.e. based on the size of the particles, allowing for a fast and direct adjustment possibility if, for instance, the determined technical application property deviates from the predetermined target technical application property for the superabsorbent material, without having to purely rely on the experience of a controller of the production process or on randomly performed quality controls. In particular, this improved production process allows to avoid waste production and to reduce the resources necessary for producing a superabsorbent material for a respective sanitary product. Moreover, the results of the property determination model can be combined with other process and feedstock data from one or more previous or subsequent steps in the production process to determine optimal process adjustments necessary to obtain or maintain a given target performance of the superabsorbent material.

Generally, the apparatus can refer to any general or dedicated computing device adapted to perform the functions of the apparatus, for example, by executing a respective computer program. In particular, the apparatus can be realized in any form of soft- and/or hardware that causes the general or dedicated computing device to perform the functions as defined above. Moreover, the apparatus can be realized in form of a standalone device, for instance, in form of a dedicated hardware, or by being provided on a respective computer system of a user, but can also be realized in form of a network of computers or processors, for instance, in a shared computation regime like cloud computing, network computing, etc. in which more than one computer or processor can provide the functions of the apparatus.

Generally, the apparatus is adapted for determining a technical application property of a superabsorbent material. Superabsorbent materials are materials that can absorb and retain large amounts of aqueous liquid relative to their own mass. For example, for deionized and distilled water a superabsorbent material can absorb up to 1000 times its own weight. For practical applications in hygiene a superabsorbent material absorbs at least 15 g/g, typically at least 20 g/g, preferably at least 25 g/g and most preferably at least 30 g/g, but not more than 120 g/g, preferably not more than 100 g/g, more preferably not more than 80 g/g, most preferably not more than 60 g/g of a 0.9 wt. % saline solution (NaCl) in the absence of external pressure, e-g—in the tea bag method CRC. Absorption by a superabsorbent polymer is particular useful as compared to conventional absorbers like cellulose fluff because even under use pressure in the hygiene application the absorbed liquid will not be released, and the skin of the wearer will be kept dry. In most cases, the superabsorbent material comprises a superabsorbent polymer (SAP) often provided in form of a plurality of particles forming the superabsorbent material. A superabsorbent polymer is typically comprised of hydrophilic, and ionic group carrying polymer chains with high molecular weight and these polymer chains are interconnected to render the superabsorbent polymer water insoluble. The ionic groups are typically —COOH, but can also be based on sulfur (SO₃H) or phosporous as a provider of acidity. These groups are partially neutralized to achieve a skin-friendly pH on the wearer's skin, typically pH=4.0-7.5, preferably pH=5.0-6.5. As means of neutralization any alkali metal based neutralization agent (Li, Na. K, Rb, Cs) can be used but preferably Na is used in hygiene applications. Neutralization agents typically used are alkali metal salts of the hydroxides, carbonates, and hydrogencarbonates and mixtures thereof. Examples for such superabsorbent polymers are cross-linked poly(meth)acrylic acid sodium salt, cross-linked poly-itaconic acid sodium salt, polyacrylamide copolymer, ethylene maleic anhydride copolymer, cross-linked carboxymethyl cellulose, cross-linked starch derivatives and carboxymethyl starch, polyvinylalcohol-grafted or starch-grafted cross-linked partially neutralized polyacrylic acid salts, polyvinyl alcohol copolymers, cross-linked polyethylene oxide, etc. Also, copolymers of acrylic acid, maleic acid, itaconic acid can be used and may be combined with copolymerized non-ionic hydrophilic or hydrophobic monomers. Preferable are partially neutralized cross-linked poly-acrylic acid and poly-itaconic acid salts as well as their copolymers. More preferable are partially neutralized cross-linked poly-acrylic acid and poly-itaconic acid salts as well as their copolymers of which the raw materials have been derived from biological sources, e.g. plants, microbes, algae, fungi.

Most preferable are partially neutralized cross-linked poly-acrylic acid and poly-itaconic acid salts as well as their copolymers and superabsorbent production processes that produce the inventive superabsorbent polymer with a carbon footprint as low as possible.

The technical application property can refer to any technical application property that is related to the superabsorbent characteristics of the superabsorbent material. Preferably, the technical application property refers to at least one of an absorption capacity, or swelling kinetics and permeability. An absorption capacity can be determined as any of a centrifuge retention capacity (CRC), a free swelling capacity (FSC), and an absorption capacity against pressure (AAP). Generally, CRC and FSC are determined without external pressure, i.e with 0.0 psi. in both cases the higher the respective CRC or FSC value the more fluid can be absorbed by a particle. A swelling kinetics can refer to any of VAUL, T20 und Vortex. For irregular shaped, rough surface particles these three quantities are correlated. For particles with a regular, smooth surface or round shape the Vortex may not be correlated with the other quantities. Generally, the faster the swelling of the particle the smaller is the respective value of any of the quantities. The permeability (SFC) is correlated nonlinearly with the CRC-capacity. Generally, the higher the permeability value the better the fluid is distributed in a particle convolute. These technical application properties are examples of different categories of technical application properties. The CRC can be interpreted as the mere absorption capacity of the superabsorbent material, the SFC can be interpreted as the mere permeability of the open pores in the swollen gel bed of the swollen superabsorbent material, the FSC, or AAP can be interpreted as absorbent capacities of the superabsorbent material under defined external pressure conditions and do each comprise a contribution from the swollen superabsorbent material and a contribution from the partially or completely liquid filled pores in the resulting gel-bed (interstitial liquid), and the T20, Vortex, and VAUL characteristic swelling time can be interpreted as kinetic swelling speed parameters describing the rate of swelling and may include additional effects other than absorption rate—for example particle morphology and surface stickiness can affect these measurements. The rate at which liquid is absorbed into the swelling superabsorbent material particle is defined as absorption rate. These properties and respective methods for determining the properties are further described below. Various other forms of properties or methods for determining respective properties exist and can also be used as technical application property according to the present invention. Other methods may differ, for example, in equipment dimensions, e.g. smaller or larger AAP-cells or permeability-cells, in the handling techniques, in a test liquid, e.g. water, artificial urine instead of saline, in the swelling times, e.g. 1, 3, 5, or 10 min instead of 30 min. Generally, the selection of the technical application properties can depend on the respective intended application, for instance for hygiene applications on the respective consumer needs. A particular useful method for measuring a technical application property is described in WO 2021/001221 which allows to determine time dependent swelling profiles of superabsorbent polymers at varied external pressures. In this way a characteristic fingerprint-curve is obtained that can be used to define the target technical application property. Particularly useful is an inline analytics method as disclosed in WO 2020/109601 which enables to determine technical application properties by Raman-spectra analytics inside the production process. Combination of such an inline technique with an inline particle size determination enable highly efficient use cases with the present invention.

Typically, absorption capacity, swelling kinetics and permeability all require optimization towards the needs in a hygiene article. Hygiene articles comprise several technical elements that are designed to manage fluid acquisition and distribution as well as superabsorbent polymer for irreversible liquid storage. These elements need to collaborate with each other and the superabsorbent polymer to achieve fast liquid acquisition, wide distribution to utilize the hygiene article's full storage capacity, and the superabsorbent polymer must quickly absorb the liquid to redry the hygiene article under use conditions. Superabsorbent polymer capacity and swelling kinetics depend on various factors, for instance, formulation, process conditions, e.g. in the gel-drying step, and in particular particle size and shape. As a consequence optimization of one aspect, e.g. capacity often affects the other aspect in the opposite direction, e.g. swelling kinetics. Hence, there is a need to not only find the optimum between capacity and swelling kinetics for a given hygiene article design but also the optimum settings in the production process for such superabsorbent polymer.

The superabsorbent material is provided in form of superabsorbent particles comprising a superabsorbent polymer. The size of the superabsorbent particles for most applications lies preferably between 100 μm and 850 μm. However, for some applications also larger or smaller superabsorbent particles can be suitable. Lately, narrower particle size distributions have been required for some hygiene applications which are much more difficult and costly to produce and challenging to optimize their technical properties, for example with a lower particle size of 100, 150, 200, or 250 μm and an upper particle size of 700 μm, 600 μm, or 500 μm.

In particular, each superabsorbent particle comprises a) an interconnected core and b) a surface cross-linked shell with a higher connectivity than the core. In this context, the terms “interconnected”, “connectivity” mean that the polymer chains of the core- or shell-part of the superabsorbent polymer particle are physically entangled, ionically or covalently cross-linked so that the respective parts of the superabsorbent particles are water-swellable but not water-soluble. A combination of such methods to interconnect the polymer chains is possible and typically found in superabsorbent particles. Ionic cross-linking can be achieved by multivalent cations, practical examples are Mg²⁺, Ca²⁺, Sr²⁺, Al³⁺, Ti⁴⁺, Zr⁴⁺. Covalent cross-linking can be achieved by addition of polymerizable di- or polyfunctional ethylenically unsaturated cross-linkers to the monomer mixture. Alternatively, this functionality can also be provided by groups that can undergo esterification or transesterification. Mixed functionalities in one molecule are also possible. For surface-cross-linking the same compounds as described above are useable. Typically, ionic cross-linkers and covalent cross-linkers are used in combination. Examples for core- and surface-cross-linking are described in WO 2019/197194 which is incorporated in here by reference. In the present invention it is understood that the shell and the core of the superabsorbent particles are linked to each other by physical entanglement or ionic or preferably by covalent cross-linking. Such a core-shell structure may break up the surface-shell of the superabsorbent particle when swelling but due to the connectedness of the shell to the core it will continue to exert physical forces onto the swelling core even if the shell is completely broken. In particular, the superabsorbent particles refer to a post cross-linked superabsorbent polymer particle. Such a post cross-linked superabsorbent polymer particle comprises a cross-linked and thus interconnected core and is then provided in a post cross-linking process with a shell comprising a higher connectivity than the core while this shell is covalently bound to its underlying core. Optionally, a such provided superabsorbent particle can also be provided with an additional non-superabsorbent coating. Non-limiting examples of such coatings are polymers or polymer films to improve flowability or damage stability, powder coatings to prevent caking (silica, alumina, clay or other inorganic powders in their dry or hydrated forms), additives to prevent ageing or discoloration, additives that combat malodour, or functional coatings that react with the surface like Ca²⁺—, Mg²⁺, A³⁺— and Zr⁴⁺— salts or their soluble hydroxides as for example published in WO 2019/197194.

The receiving interface is configured for receiving a particle size of superabsorbent particles of the superabsorbent material. In particular, the receiving interface can be configured to interface with a storage unit on which a superabsorbent particle size is already stored. However, the receiving interface can additionally or alternatively be configured to interface with a user input unit such that a user can provide a superabsorbent particle size. Moreover, the receiving interface can additionally or alternatively also be configured to interface with a measurement unit, for example, a sensor, configured to measure a particle size or particle size distribution of a superabsorbent material, for example, during a production process. Generally, the particle size of the superabsorbent particle can be received in form of any quantity indicative of a volume of the superabsorbent particles, for example, can be received as a volume, a radius, a diameter, etc. of the superabsorbent particle. Moreover, the particle size refers to a dry state of the superabsorbent particle, and thus to a state in which the superabsorbent particle has no contact to an absorbable fluid. In the dry state the superabsorbent particles comprise a moisture content of less than 25 wt. %, typically less than 20 wt. %, preferably less than 15 wt. %, more preferably less than 10 wt. %, even more preferably less than 5 wt. %, and most preferably less than 3 wt. %.

The one or more processors are then further configured to utilize a property determination model for determining the technical application property of the superabsorbent material based on the particle size. In particular, the property determination model is realized as a data-driven model that has been parameterized, for example, based on historical measurement data, such that it is adapted to determine a technical application property of a superabsorbent particle based on the size of the particle. In particular, the term “data-driven” defines that the model is mainly based on respective data input and not, for instance, on intuition, personal experience or knowledge. The property determination model can, in particular, be realized as any machine learning based model that is based on known machine learning algorithms, like neural networks, regression models, classification algorithms, etc. A white-box model evaluated with a non-linear regression optimizer is particularly useful in the present invention. Such a machine learning model can be used as part of a comprehensive process control model—taking into account other formulation and process parameters—using other machine learning and artificial intelligence algorithms. Generally, the property determination model comprises one or more model parameters that can be determined based on respective training data. The parameterization of the property determination model is thus a determination of the values of the respective one or more model parameters based on the training data in a model training process. In particular, the determination model is parameterized such that it can determine a technical application property of a superabsorbent particle based on the size of the particle. Generally, for parameterizing the property determination model accordingly, respectively known training methods for parameterizing a given model can be utilized. In particular, optimization methods can be utilized to find an optimal fit of model parameters to the respective training data. For the training of the property determination model preferably historical data can be utilized as training data, for example, historical data comprising measurement data from respective measurements of technical application properties of superabsorbent particles or data derived from known physical relations of the technical application property and respectively measurable characteristics of the superabsorbent particle. In particular, the historical data comprises for a plurality of different particle sizes of a superabsorbent particle corresponding to one or more technical application properties.

Since the production process of a superabsorbent particle can have a high influence on the specific composition of the superabsorbent particle, for example, on the specific interconnectivity of the core and the shell of the superabsorbent particle, it is preferred that a respective historical data set for a specific production process for respectively produced superabsorbent material is utilized for parameterizing the property determination model. This allows the property determination model to provide a very accurate determination of the technical application property of the superabsorbent material produced in the specific production process. However, in other embodiments, the property determination model can also be trained with a more general historical data set comprising data for superabsorbent materials utilizing different production processes, wherein the property determination model can in such a case then learn to differentiate between superabsorbent materials produced in different production processes and the respective production process can be provided as further input to the property determination model. Moreover, the property determination model can be trained such that it can determine one technical application property of a superabsorbent material, but can also be trained to predict more than one technical application property of a superabsorbent material.

The output interface is then configured for generating control data based on the determined technical application property. In particular, the output interface can be configured to interface with a user interface, wherein the control data can then be generated for controlling the user interface for providing the determined technical application property. However, the control data can refer to other applications and can be provided on the user interface such that, for example, a user can then decide whether or not to implement the control data or such that the user can arrange for amendments of the control data. However, the output interface can additionally or alternatively be configured to interface with other computing systems or production systems. For example, the output interface can be configured to interface with a production management system controlling a production of the superabsorbent material to provide the control data to the production management system, and/or can be configured to directly interface with a production system configured to produce the superabsorbent material to provide the control data directly to the production system. In a preferred embodiment, the generated control data comprises a manufacturing specification for controlling a manufacturing of the superabsorbent material. In particular, it is preferred that the manufacturing specification comprises information indicative of a size of the superabsorbent particles to be produced. The manufacturing specification can further comprise additional information on the superabsorbent particles relevant for the production, for example, can comprise one or more process parameters indicative for the production process of the superabsorbent particles, a recipe indicative of substances or materials utilized during a production process, etc. Preferably the manufacturing specification is provided in form of a control sequence of a production system, such that based on the control data the superabsorbent material can be directly produced. Generally, the control data can be generated, for example, based on predetermined rules determining which control data is generated based on which determined technical application property.

In a preferred embodiment, the utilized property determination model is further parameterized based on a core size and a shell size of the superabsorbent particle. Surprisingly, it has been found by the inventors that further parameterizing the property determination model based on a core size and a shell size of the superabsorbent particle allows for a particularly accurate determination of the technical application property. Moreover, parameterizing the property determination model further based on a core size and a shell size of the superabsorbent particle allows to separate the respective influence of each of these parameters on the technical application property. Generally, the core size and the shell size of a superabsorbent particle depend on the particle size, i.e. the penetration depth of substances used for the post cross-linking process is substantially the same for all particle sizes such that the size, i.e. volume, of the shell and the core depend mainly on the size of a particle. However, the general penetration depth of the post cross-linking substances and thus the thickness of the shell relative to the core depend on the utilized cross-linking procedure, for example, the utilized substances, pressure conditions, utilized additives, temperatures, etc. Thus, further separating the influence of the core and shell size on the technical application property allows not only for utilizing the size of the superabsorbent particles for controlling the technical application properties, but also allows to determine the influence of the shell size and core size on the technical application property and thus allows to also optimize the post cross-linking procedure for the superabsorbent particles with respect to the technical application property.

Preferably, the parameterizing of the utilized property determination model comprises determining performance parameters quantifying a contribution of the core size and the shell size, respectively, of a superabsorbent particle to the technical application property. Utilizing a respective training data set that comprises for a plurality of superabsorbent particle sizes corresponding core sizes, shell sizes and technical application properties, allows to parameterize a property determination model such that the influence of the core size and the shell size on the technical application property can accurately be quantified by determining the performance parameters. Preferably, the utilized property determination model is based on the following relation between the technical application property and a size of a superabsorbent particle

$${var}_{Particle} = \frac{{V}_{shell} {var}_{shell} + {V}_{core} {var}_{core}}{{V}_{shell} + {V}_{core}}$$

wherein Vₛₕₑₗₗ is a volume of the shell, and V_{core} is a volume of the core of the superabsorbent particle, wherein the volume of the shell and the volume of the core of the superabsorbent particle depend on the size of the superabsorbent particle, and wherein varₛₕₑₗₗ and var_{core} are the performance parameters quantifying the contribution of the core size and the shell size, respectively, and are determined during the parameterization of the property determination model, and wherein var_{Particle} is indicative of the measured technical application property.

In an embodiment, the received particle size is a particle size distribution of the superabsorbent particles of the superabsorbent material, wherein the one or more processors are further configured to a) determine based on the particle size distribution one or more particle size classes from predetermined particle size classes for which particles with respective sizes are present in the superabsorbent material, b) determine a technical application property for the determined particle size classes and c) determine an overall technical application property of the superabsorbent material based on the determined technical application properties for the respective determined particle size classes and based on the particle size distribution. Generally, the particle size distribution of a superabsorbent particle can be received in form of any data information that is indicative of the particle sizes that are present in a statistically relevant sample of the superabsorbent material. For example, the particle size distribution can be provided in the form of a list of all sampled superabsorbent particles and corresponding sizes of the superabsorbent particles. However, the particle size distribution can also directly be provided in the form of a class distribution indicating for a plurality of particle size classes the amount of particles present in the respective class in a statistically relevant sample. Generally, a particle size class refers to a particle size range and is defined by a smallest particle size and a largest particle size defining the particle size range. The respectively predetermined particle size classes can then refer to the already utilized particle size classes if the particle size distribution is provided in the form of a class distribution, wherein in this case the determination of whether particles are present in a predetermined particle size class amounts to determining whether an amount of particles greater than zero is indicated by the particle size class distribution in a respective particle size class. However, the predetermined particle size classes can also be independent of any previously utilized particle size classes for providing a particle size class distribution. In this case, respective statistical methods can be utilized to determine for which of the predetermined particle size classes particles are present in the superabsorbent material. Moreover, if a list of particle sizes is provided as particle size distribution, the predetermined particle size classes can be utilized to sort the particle sizes accordingly and to determine for which predetermined particle size class at least one particle is present. Preferably, the particle size distribution, for instance, of water-absorbent polymer particles, can be determined by the EDANA recommended test method No. WSP 220.3 (11) “Particle Size Distribution”. Also, optical methods, for example, laser diffraction, photographic analysis, etc., can be favourably utilized, preferably, calibrated against the EDANA or corresponding ISO-test method based on a screening analysis. Such calibration may depend also on other particle properties except the particle size and is therefore carried out specifically for each product grade. In the present invention such calibrated methods are particularly useful as they can be used inline of the production process at one or more locations and provide the necessary particle size information in real time.

The technical application property for the determined particle size classes is then determined by utilizing the property determination model for a respective particle size falling within the predetermined particle size classes. Generally, the technical application property can be determined by providing at least one particle size falling in a predetermined particle size class to the property determination model and utilizing the determined technical application property as technical application property for all sizes falling within the determined particle size class. However, also the smallest and the largest size of particles falling within the respectively determined particle size class can be utilized as input to the property determination model and the respectively determined technical application properties can be statistically combined, for instance, by averaging to determine a technical application property representing the respective determined particle size class. However, also other statistical methods can be utilized accordingly. The overall technical application property can then be determined based on the determined technical application properties for the respective determined particle size classes and based on the particle distribution. In particular, the amount of particles falling within a respective determined particle size class is taken into account in determining the overall technical application property. For example, a weighted averaging can be utilized for determining the overall technical application property based on the determined technical application properties, wherein the weights of the weighted averaging are determined based on the amount of particles of the particle distribution falling within a respective particle size class. For example, if more particles fall within a particle size class, the respective technical application property can be weighted higher than a technical application property corresponding to a particle size class with less particles. Moreover, also other known or learned relations can be taken into account, for instance, in the weights for determining the overall technical application property. For exampie, it can be determined that bigger particles have generally a higher influence on an overall technical application property than smaller particles. In this case, the weights for particle size classes with bigger particles can be provided higher with respect to the weights of particle size classes with lower size particles. The control data is then preferably generated based on the determined overall technical application property.

Typically, for absorption capacities with and without external pressure a weighted average can be utilized to predict the overall product properties from the individual particle size classes. For performance critical properties like swelling kinetics and liquid permeability this is not the case and rather complex mixing properties are found for blends of superabsorbent particles with different properties—for example as described in WO 2019/137833. This is due to the fact that for such blends not only the size-class-specific properties are relevant but also complex mixing phenomena based on the amount of particles present in the different size-classes show significant impact on the overall performance. While more finer particles can be beneficial to achieve fast liquid absorption, they can act antagonistic in terms of liquid distribution as they quickly block fluid conducting pores. To predict such properties a weighted average value of the size-classes may be used that does not treat all particle sizes equally and can depend on the number of particles in each class. In the present invention, the weights are preferably experimentally determined by first measuring the superabsorbent polymer's overall performance properties and then after classifying into the respective discrete size classes by determination of the properties for each class. Mixing the respective classes in varied amounts with each other will allow conclusions towards the required weights. For such mixing one can use a design of experiments.

In an embodiment, the received particle size is provided as a starting particle size and wherein the receiving interface is further adapted to receive a target application property, wherein the processor is further adapted to iteratively determine a target particle size such that the superabsorbent material meets the target application property within predetermined limits, wherein the iteration comprises a) determining in each iteration step a technical application property, b) comparing the determined technical application property with the target technical application property, and c), based on the comparison, provide an amended particle size, or determine the particle size as the target size. In case the particle size is provided in form of a particle size distribution the target technical application property refers to a target overall technical application property and the overall technical application property can be determined as described above. The control data is then generated based on the target size or target size distribution. In particular, the control data can in such a case be generated such that a production system for producing the superabsorbent particles produces the superabsorbent particles with the target size or the target size distribution.

Additionally, or alternatively, the one or more processors can also be adapted to determine a deviation of the determined technical application property from the target technical application property and the control data can then be generated based on the deviation. Thus in this case the iteration can be omitted and for such cases the control data can directly be generated based on the deviation. For example, predetermined rules can be utilized to generate the control data based on the deviation. For example, if a deviation is determined between the determined technical application property and the target technical application property, the control data can be generated to amend the production process, for instance, such that the particle size is decreased or increased about a predetermined amount. After the production of the superabsorbent particles with the decreased or increased size the technical application property of the superabsorbent particles can then again be determined and again be compared with the target technical application property. This process can then be repeated until the determined technical application property meets the target technical application property.

In a further aspect of the invention, an apparatus for determining an overall technical application property of a superabsorbent material is presented, wherein the superabsorbent material is provided in form of superabsorbent particles comprising a superabsorbent polymer provided in form of i) an interconnected core and ii) a surface cross-linked shell with a higher connectivity than the core, wherein the apparatus comprises a) a receiving interface receiving a particle size distribution of the superabsorbent particles of a superabsorbent material, b) one or more processors configured to i) determine based on the particle size distribution one or more particle size classes from predetermined particle size classes for which particles with respective sizes are present in the superabsorbent material, ii) utilize a property determination model for determining a technical application property of the superabsorbent material for the determined particle size classes, wherein the property determination model is a data-driven model that has been parameterized such that it is adapted to determine a technical application property of a superabsorbent particle based on the size of the particle, and iii) determine an overall technical application property of the superabsorbent material based on the determined technical application properties for the respective determined particle size classes and based on the particle size distribution, and c) an output interface for generating control data based on the determined overall technical application property.

In a further aspect of the invention, an interface apparatus for providing an interface for determining a technical application property of a superabsorbent material is presented, wherein the interface apparatus comprises a) an interface input unit for interfacing with the apparatus as described above for providing a particle size to the apparatus as described above, and b) an interface output unit for processing control data generated by the apparatus as described above based on the particle size.

In a further aspect of the invention, a training apparatus for parameterizing a property determination model is presented, wherein the training apparatus comprises a) a receiving interface for receiving historical training data comprising a plurality of particle sizes for superabsorbent particles of a superabsorbent material and corresponding one or more measured application properties of the superabsorbent material, b) one or more processor configured to utilize the received historical training data for parameterizing a property determination model such that the parameterized property determination model is adapted to determine a technical application property of the superabsorbent material based on a particle size, and c) an output interface for outputting the parameterized property determination model.

In a further aspect of the invention, an optimization apparatus for determining a target superabsorbent material comprising a target technical application property is presented, wherein the superabsorbent material is provided in form of superabsorbent particles comprising a superabsorbent polymer provided in form of i) an interconnected core and ii) a surface cross-linked shell with a higher connectivity than the core, wherein the apparatus comprises a) a receiving interface configured for receiving the target technical application property for the target superabsorbent material and a particle size of potential superabsorbent particles of a superabsorbent material, b) one or more processors configured to i) utilize a property determination model for determining the technical application property of the potential superabsorbent material based on the particle size, wherein the property determination model is a data-driven model that has been parameterized such that it is adapted to determine a technical application property of a superabsorbent particle based on the size of the particle, and ii) compare the determined technical application property with the target technical application property and determine I) the particle size as the target particle size if the predicted technical application property lies within a predetermined range around the target application property, or II) an amended particle size and repeat the determination of the technical application property using the amended particle size, if the predicted technical application property lies outside a predetermined range around the target application property, and c) an output interface configured for generating a control signal based on the target particle size. Preferably, the received potential particle size is a particle size distribution of the superabsorbent particles of the target superabsorbent material. As described above with respect to the apparatus the one or more processors can then further be configured to a) determine based on the particle size distribution one or more particle size classes from predetermined particle size classes for which particles with respective sizes are present in the superabsorbent material, b) determine a technical application property for the determined particle size classes and c) determine an overall technical application property of the superabsorbent material based on the determined technical application properties for the respective determined particle size classes and based on the particle size distribution. The determined overall technical application property can then be compared with the target technical application property and it can be determined that the potential particle size distribution is the target particle size distribution or an amended particle size distribution can be determined as new potential particle size distribution. For example, the amounts of particles in one or more particle size classes can be amended. However, in an embodiment a target particle size distribution can also be determined analytically, by a) providing one or more potential target particle size classes, b) determining a technical application property for each of the potential target particle size classes, and c) determining a target amount of particles in each potential target particle size class by optimizing the amount of particles in each particle size class such that the overall technical application property meets the target technical application property.

In further aspect of the invention, an interface method for providing an interface for determining a technical application property of a superabsorbent material is presented, wherein the interface method comprises a) providing, via an input interface, a particle size to the apparatus as described above, and b) processing, via an output interface, control data generated by the apparatus as described above based on the particle size.

In a further aspect of the invention, a training method for parameterizing a property determination model is presented, wherein the training method comprises a) receiving historical training data comprising a plurality of particle sizes for superabsorbent particles of a superabsorbent material and corresponding measured one or more application properties of the superabsorbent material, b) utilizing the received historical training data for parameterizing a property determination model such that the parameterized property determination model is adapted to determine a technical application property of the superabsorbent material based on a particle size, and c) outputting the parameterized property determination model.

In a further aspect of the invention, an optimization method for determining a target superabsorbent material comprising a target technical application property is presented, wherein the superabsorbent material is provided in form of superabsorbent particles comprising a superabsorbent polymer provided in form of i) an interconnected core and ii) a surface cross-linked shell with a higher connectivity than the core, wherein the method comprises a) receiving the target technical application property for the target superabsorbent material and a particle size of potential superabsorbent particles of a superabsorbent material, b) utilizing a property determination model for determining the technical application property of the potential superabsorbent material based on the particle size, wherein the property determination model is a data-driven model that has been parameterized such that it is adapted to determine a technical application property of a superabsorbent particle based on the size of the particle, c) comparing the determined technical application property with the target technical application property and determine I) the particle size as the target particle size if the predicted technical application property lies within a predetermined range around the target application property, or II) an amended particle size and repeat the determination of the technical application property using the amended particle size, if the predicted technical application property lies outside a predetermined range around the target application property, and d) generating a control signal based on the target particle size.

In a further aspect of the invention, a computer program product for determining a technical application property of a superabsorbent material is presented, wherein the computer program product comprises program code means for causing the apparatus as described above to execute the method as described above.

In a further aspect of the invention, a computer program product for training a property determination model is presented, wherein the computer program product comprises program code means for causing the training apparatus as described above to execute the training method as described above.

In a further aspect of the invention, control data generated according to the apparatus, method and/or computer program product as described above is presented.

In a further aspect of the invention, a use of the apparatus, the method according and/or the computer program as described above for determining an application property of a superabsorbent polymer is presented.

In a further aspect of the invention, a use of the apparatus, the method and/or the computer program as described above for generating a library of technical application properties of different superabsorbent polymers is presented.

In a further aspect of the invention, a use of the apparatus, the method and/or the computer program product as described above for controlling a production process of a superabsorbent material, in particular, controlling a particle size distribution, is presented.

It shall be understood that the methods as described above, the apparatuses as described above and the computer program products as described above have similar and/or identical preferred embodiments, in particular, as defined in the dependent claims. Moreover, also the training method as described above, the training apparatus as described above and the training computer program product as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereafter.

### BRIEF DESCRIPTION OF THE DRAWINGS

In the following drawings:

FIG. 1 shows schematically and exemplarily a system for producing a superabsorbent material comprising an apparatus for determining a technical application property of the superabsorbent material,

FIG. 2 shows schematically and exemplarily a flowchart of a method for determining a technical application property of a superabsorbent material and optionally controlling a production of the superabsorbent material,

FIG. 3 shows schematically and exemplarily a training apparatus for training a property determination model for determining a technical application property of a superabsorbent material,

FIG. 4 shows schematically and exemplarily a flowchart of a method for training a property determination model for determining a technical application property of a superabsorbent material,

FIG. 5 shows schematically and exemplarily a model of a swelling of a superabsorbent particle, and

FIG. 6 shows schematically and exemplarily an application of the determination of a technical application property of a superabsorbent particle.

### DETAILED DESCRIPTION OF EMBODIMENTS

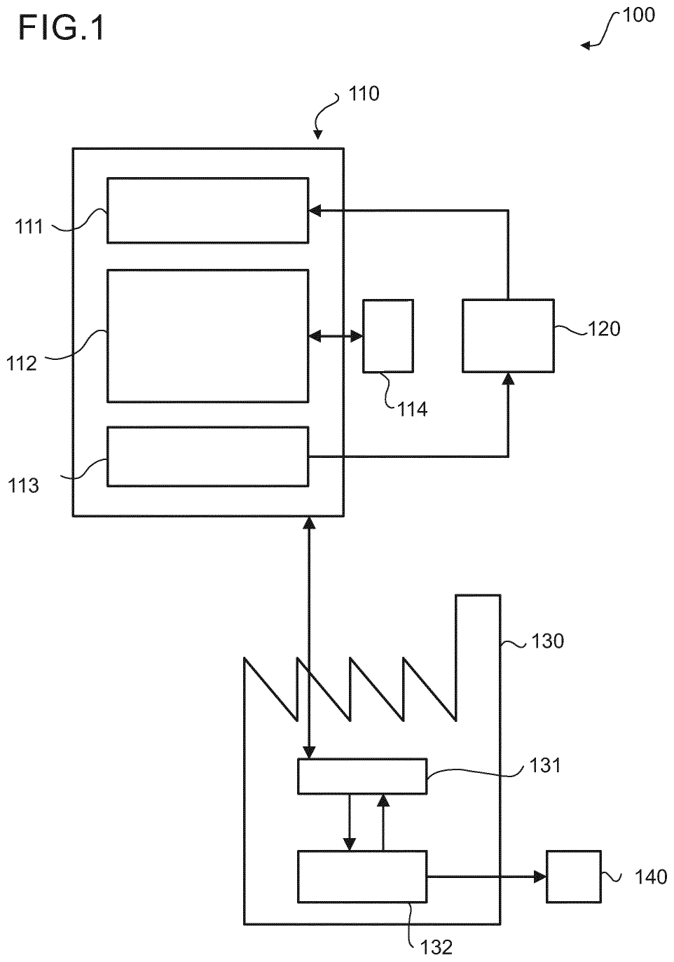

FIG. 1 shows schematically and exemplarily a system 100 for producing a superabsorbent material 140. The system 100 comprises an apparatus 110 for determining a technical application property of a superabsorbent material 140. Optionally, the system can further comprise an interface apparatus 120 for interfacing with the apparatus 110. Moreover, the system can comprise a production system 132 and a production control system 131 provided in a production plant 130 producing the superabsorbent material 140.

The apparatus 110 comprises a receiving interface 111, one or more processors 112 and an output interface 113. Generally, the apparatus is configured to determine a technical application property of a superabsorbent material. The superabsorbent material is provided in form of superabsorbent particles comprising a superabsorbent polymer provided in form of an interconnected core and a surface cross-linked shell with a higher connectivity than the interconnected core. Generally, the apparatus can be provided as a standalone device, for example, can be provided as a dedicated computing device, but can also be provided as part of a more general computing device providing additional functions. In particular, the apparatus can be provided as part of a quality control system or, for example, as part of the production control system 131.

The receiving interface 111 is configured to receive a particle size of the superabsorbent particles of the superabsorbent material. Generally, the receiving interface can be realized as any interface that allows to receive respective data indicative of the particle size. In particular, the receiving interface can be configured to provide an interface to a storage unit on which a particle size is already stored, a controlling system, like controlling system 131 of the production system 132 providing sensor measurements indicative of the particle size, or to a user interface 120 allowing a user to input a respective particle size. The particle size can refer to any quantity that allows to quantify a volume of a particle of the superabsorbent particles of the superabsorbent material. Preferably, the particle size refers to a volume of the particle or if the particle can be approximated as a spherical particle, to a radius or diameter of the particle. The particle size of the superabsorbent particles generally is provided in a dry state of the superabsorbent particles, i.e. before the absorption of a fluid into the superabsorbent particles leading to an increase of the size of the superabsorbent particles. The received particle size of the superabsorbent particles is then provided to the one or more processors 112.

The one or more processors 112 are then configured to utilize a property determination model for determining the technical application property of the superabsorbent material based on the particle size. For example, the one or more processors can be configured to access a storage unit 114 on which the property determination model is already stored. Generally, on a storage unit 114 more than one property determination model can be stored, for instance, property determination models for different superabsorbent materials produced in accordance with manufacturing specifications, for instance, using different superabsorbent polymers, different cross-linking methods and/or production parameters, can be stored. In this case, the one or more processors can then be configured to utilize respective information on the superabsorbent material, for instance, an ID of the superabsorbent material or a manufacturing specification provided for the superabsorbent material to select the respective property determination model for the superabsorbent material. Moreover, for different technical application properties, different property determination models can be stored on the storage unit 114. In this case, the one or more processors can be configured to either select all property determination models available for a respective superabsorbent material and to then apply each of the property determination models to determine all technical application properties available for the respective superabsorbent material, or based on further information on the desired technical application property, for example, provided via the user interface 120, the one or more processors can be configured to select the respective property determination model to be utilized.

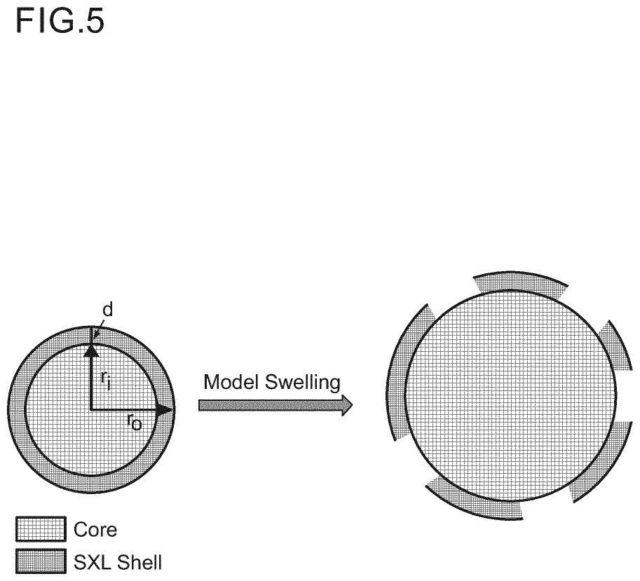

Generally, the property determination model has been parameterized such that it is adapted to determine a technical application property of a superabsorbent particle based on the size of the particle. In particular, the data-driven determination model can be any machine learning based model that allows to learn based on historical data to determine a technical application property of a superabsorbent particle based on the size of the particle. For example, the property determination model can refer to a regression model based algorithm like a neural network algorithm, a lasso algorithm, a ridge regression algorithm, a MASS algorithm or a random forest algorithm. However, the property determination model can also refer to a classifier-based model algorithm like a random forest algorithm or an SVM algorithm. A particularly preferred embodiment of the property determination model is described with respect to FIG. 5. In particular, it is preferred that the utilized property determination model is further parameterized based on a core size and a shell size of a respective superabsorbent particles in order to allow to quantify and determine the respective contributions of the core size and the shell size to the respective technical application property. This allows to store the property determination models by storing the respective performance parameters quantifying the contribution. A selection of the property determination model can then be realized by selecting the performance parameters corresponding to a respective core size and respective shell size and using these performance parameters in the property determination model.

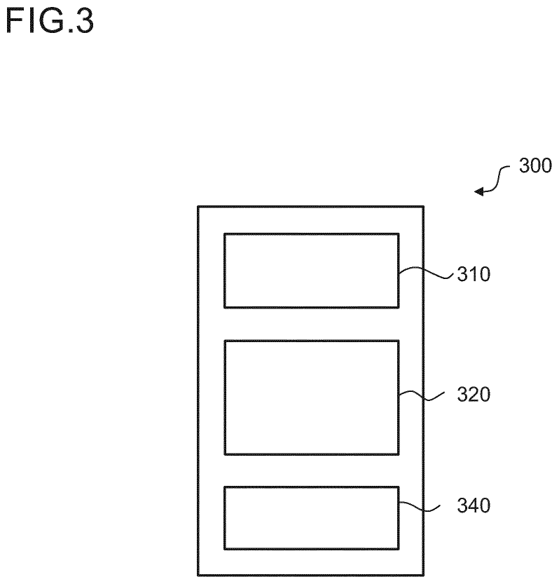

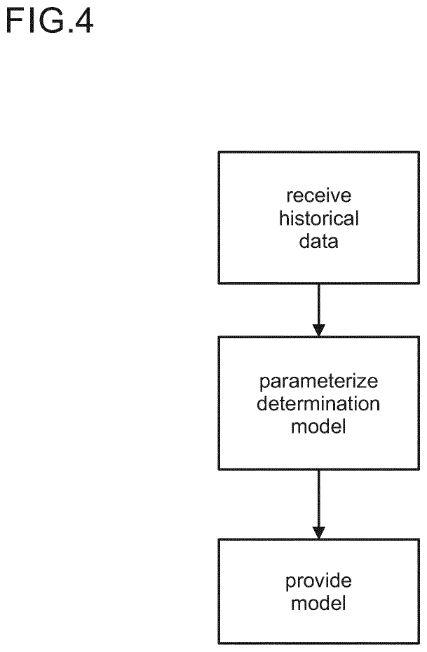

Generally, the property determination model can be trained utilizing, for example, training apparatus 300 as shown in FIG. 3 that can be configured to perform the training method as shown in FIG. 4. The training apparatus 300 as shown in FIG. 3 comprises a receiving interface 310, one or more processors 320 and an output interface 340. Generally, the training apparatus 300 can be integrated, for example, into the apparatus 110. In this case, the apparatus 110, in particular, the one or more processors 112 of the apparatus 110, can be configured, if no property determination model is available, for example, on storage 114, to instead access, for example, a storage unit storing respective historical training data and then to utilize apparatus 300 to train the respective property determination model. However, training apparatus 300 can also be provided independent of the apparatus 110 and can then be configured to provide the trained property determination models to a storage unit like a storage unit 114 to which the apparatus 110 has access.

The receiving interface 310 is configured to receive historical training data for training the property determination model. Generally, the receiving interface can provide an interface to a storage unit on which the historical training data is stored or to a measurement or sensor interface that allows to receive respective measurements that can be utilized as historical training data. The historical training data comprises at least two, preferably a plurality of particle sizes for a superabsorbent material and corresponding one or more measured application properties of the superabsorbent material. Such training data can be generated, for instance, by measurement of a respective superabsorbent material using known measurement methods for determining the technical application property and also for measuring the respective particle size of the superabsorbent material. Generally, such historical training data is often generated during a quality control of a superabsorbent material or during a design process of a superabsorbent material in which respective measurements are performed.

The one or more processors 320 are then configured to utilize the received historical training data for parameterizing the property determination model such that the parameterized property determination model is adapted to determine the technical application property of the superabsorbent material based on the particle size. For example, known machine learning, i.e. parameterizing, methods can be utilized. The output interface 340 is then configured for outputting the parameterized property determination model, for example, to a respective storage, like storage 114 or directly to an apparatus for determining the technical application property like apparatus 110. Generally, for training the property determination model a method as schematically and exemplarily shown in FIG. 4 can be utilized. For example, historical training data can be received, for example, like described with respect to the receiving interface of training apparatus 300. The respective historical training data can then be utilized to parameterize, using known training methods, a respective property determination model, as described in more detail with respect to training apparatus 300. Further, the trained property determination model can then be provided, for example, to apparatus 110.

Based on such a trained property determination model, the one or more processors 112 of apparatus 110 are then configured to determine a technical application property of the superabsorbent material based on the provided particle size. The determined technical application property and optionally also the utilized particle size is then provided to the output interface 113. The output interface 113 is then configured to generate control data based on the determined technical application property. For example, the output interface can generate control data that allow to provide the determined technical application property to the user interface 120 in order to inform a user of the determined technical application property. However, in a preferred embodiment the generated control data comprises a manufacturing specification for controlling a manufacturing of the superabsorbent material, in particular, to control the production system 132 for producing the superabsorbent material 140. The manufacturing specification can then be provided, for instance, to control system 131 to control the production system 132 to produce the respective superabsorbent particles 140.

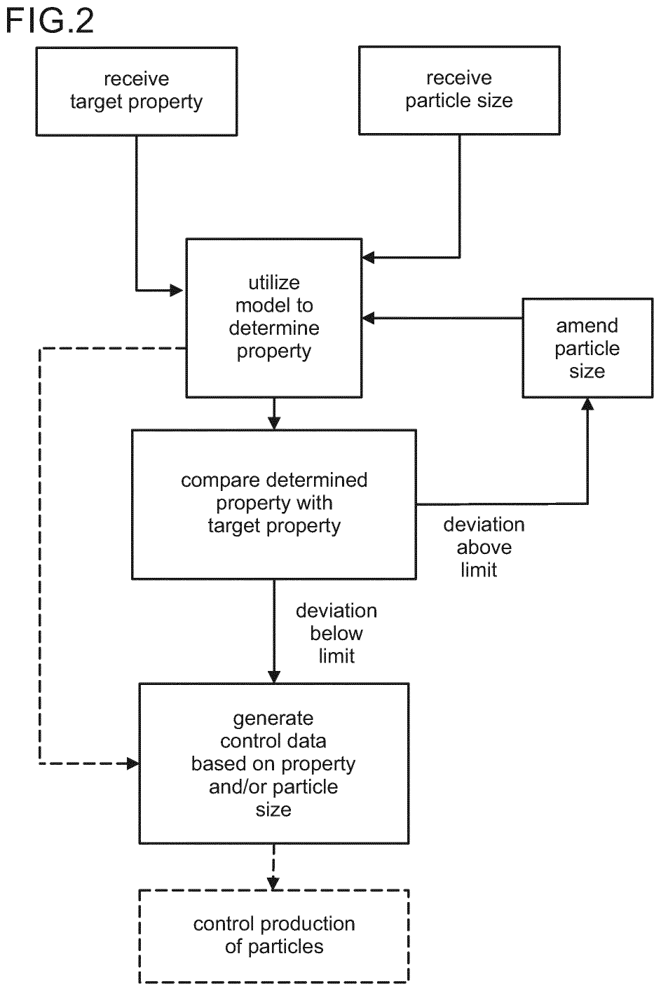

However, in a preferred embodiment, the apparatus 110 is further configured to not only determine the technical application property, but further to perform an iteration to determine a particle size that allows the superabsorbent material to provide a target technical application property. In particular, the apparatus can be configured, for example, to perform the method as exemplarily and schematically shown in the flow chart of FIG. 2. Generally, the method as shown in FIG. 2 can be performed in the context of a design of product process in which a superabsorbent product with respective predetermined target technical application properties is designed and a respective superabsorbent material has to be found to provide the respective target technical application properties. In this case, the iteration is only based on the respective target technical application property and no further measurements are provided as part of the feedback loop. However, the method can also be applied in the context, for instance, of the system 100 shown in FIG. 1 for providing controlling feedback to the production system 132 in order to produce a superabsorbent material 140 with the respective desired technical application property. In this case, for example, measurements of the particle size of a current batch of superabsorbent material can be utilized as input into the iteration and the production process can be amended concurrently until a measured particle size is reached that provides the respective desired technical application property.

Generally, in the method described in FIG. 2 a target technical application property is received that refers to a technical application property that should be met by the respective superabsorbent material. Moreover, the method comprises receiving a respective particle size, for instance, as an arbitrary starting particle size or as a measured particle size provided by a current batch of produced superabsorbent material. Moreover, as will also be described in the following, the provided particle size can also be a particle size distribution of a statistically relevant sample of the superabsorbent material. In many cases, it is not possible in the production of the superabsorbent material to produce particles of only one size. Thus, in many cases the production yields a superabsorbent material comprising a distribution of different particle sizes. Such a particle size distribution can be determined, for instance, by measuring a respective statistically relevant amount of superabsorbent material, to determine the size of the respective superabsorbent particles. The such determined particle size distribution can be provided, for example, in form of a list of all measured particle sizes. However, the particle size distribution can also be provided, for instance, in form of a histogram determining different particle size classes referring to respective particle size ranges and determining how many particles are found in the respective amount of superabsorbent material in each of the respective particle size classes. Measuring a particle size distribution in form of a histogram can, for example, be performed by using differently sized sieves and measuring the amount of particles being captured by each sieve. The size of the sieves then provide the boundaries of the respective particle size classes.

In a next step, the property determination model is utilized to determine a technical application property for the respective superabsorbent material. If only one particle size is provided, the technical application property for the superabsorbent material is determined, as already described above, for instance, with respect to apparatus 110. In case the particle size is provided in form of a particle size distribution for the determination of the overall technical application property of the particle size distribution, the different particle sizes have to be taken into account. For example, if a list of particle sizes is provided as particle size distribution, for each of the particle sizes the property determination model can be utilized to determine a respective technical application property, wherein the overall technical application property of the superabsorbent material can then be determined, for instance, by averaging over the plurality of determined technical application properties. Optionally, also weighted averaging can be utilized, for example, if it is known that certain particle sizes have a higher influence on the overall technical application property than other particle sizes. However, for most cases, it is not necessary to determine a technical application property for exactly each particle size in order to provide a suitable accuracy. In fact, in most cases a measurement of the particle size will not yield in an exact particle size but will yield a particle size distribution, for instance, in form of a histogram that can easily be measured utilizing sieves, wherein for each sieving step the resulting amount of particles is successively measured. In this case, it is preferred that particle size classes are utilized and the technical application property is determined for each particle size class. Generally, the utilized property determination model can accordingly be trained already utilizing the same particle size classes. However, the property determination model can also be trained without utilizing particle size classes. If the property determination model is already trained based on particle size classes, then it only has to be determined based on the provided particle size distribution for which classes at least one particle is present in the particle size distribution. For the such determined particle size classes the property determination model can then be utilized to determine the technical application property. Also, in the case that the property determination model has been trained based on specific particle sized, i.e. without utilizing particle size classes, respective particle size classes can be utilized. For example, in this case, the particle size classes that are already provided in the particle size distribution can be utilized or other particle size classes can be predetermined. It is then also determined based on the particle size distribution in which particle size classes at least one particle can be found in the superabsorbent material. Then, one or more particle sizes representing a particle size class can be determined. For example, a particle size lying in the middle of the particle size class can be utilized as representing the particle size class. However, also the smallest and the largest particle size of a particle size class can be utilized for representing the particle size class. Based on the represented particle size values a technical application property can then be determined utilizing the property determination model. If more than one value is utilized for representing a particle size class, it is preferred that statistical methods are utilized to determine based on the technical application properties determined for the more than one size value representing the particle size class a technical application property that corresponds to this particle size class. For example, an average value for the determined technical application properties can be utilized. Thus, also in this case for each particle size class a technical application property is determined that corresponds to this particle size class. Based on all determined technical application properties for all particle size classes in which at least one particle is present in the superabsorbent material, then an overall technical application property can be determined. For example, respective statistical methods can be utilized like averaging or in particular weighted averaging. Moreover, known relations of the different particle size classes and their contribution to a respective technical application property can also be taken into account. For example, if larger particles have a higher influence on the technical application property than smaller particles during an averaging process, the technical application properties corresponding to particle size classes of larger particles can be provided with a higher weight than the technical application properties corresponding to particle size classes of smaller particles. Moreover, when determining the overall technical application property, also the particle size distribution is taken into account. In particular, the relative amount of particles in a particle size class compared with particles in other particle size classes is taken into account for determining the overall technical application property. For example, in an averaging process, particle size classes comprising more particles can be weighted higher than particle size classes comprising fewer particles. In particular, the weights can be chosen based on the respective percentage of particles in a respective particle size class with respect to the overall number of particles in the respective sample. Accordingly, also in case of a particle size distribution the above described method allows to determine a very accurate overall technical application property of the superabsorbent material comprising this particle size distribution.

In the next step, the such determined technical application property, in case of a particle size distribution, overall technical application property, is then compared with the target technical application property. In particular, it is determined whether the determined technical application property deviates from the target technical application property. If the deviation between the determined technical application property and the target technical application property lies above a predetermined limit, i.e. if the determined technical application property does not meet the target technical application property within these predetermined limits, a next iteration step can be initiated. In particular, in the next iteration step the particle size can be amended, for instance, increased or decreased. In case of a particle size distribution, the respective particle size distribution is amended, for example, by changing the respective amount of particles in the different particle size classes. This step can refer to a purely computer implemented step. However, in case of the controlling of a production process, this step can also refer to providing control data that controls the production process of the superabsorbent material such that the particle size produced is amended respectively. In this case, also as feedback loop the amended particle size can again be measured, since for respective production processes it is often not possible to accurately ensure that the particle size is amended as planned. In this case then the measured amended particle size is utilized in the next iteration step. Based on the respectively amended particle size or particle size distribution, then again the property determination model is utilized as described above to determine a technical application property.

These iteration steps can be performed and repeated until either a predetermined abortion criterion is reached, for instance, a predetermined number of iteration steps have been performed, or the comparison results in a deviation below the predetermined limit, i.e. until a technical application property is determined that meets the target technical application property within the predetermined limits. In this case, respective control data is generated based on the technical application property and/or the such determined target particle size. In particular, it is preferred that control data is generated that comprises a manufacturing specification indicating, in particular, the target particle size or particle size distribution that should be reached during the production of the superabsorbent particles. The respective manufacturing specification can then be utilized optionally to control the respective production of the superabsorbent particles. However, if the method is utilized fordirectly controlling a production process, the control data in this case can also be simply indicative of the fact that with the current production settings, for instance, process parameters of the production process of the superabsorbent particles, the respective target technical application property is reached such that these production parameters should not be amended further.

In an optional embodiment, the control data, in particular, the manufacturing specification, can also be directly determined based on the determined technical application property without further iterative steps for determining a target technical application property. This can be in particular the case if a target technical application property is directly met or the control data can refer to providing the determined technical application property to a user interface.

In a preferred embodiment of the property determination model, the property determination model, as will be described with respect to FIG. 4, is parameterized further based on a core size and a shell size of the superabsorbent particle. Generally, the core size and the shell size of the superabsorbent particle depend on the particle size. However, the core size and the shell size further depend on process parameters of the production process of the superabsorbent particle, in particular, on process parameters having influence on the cross-linking process and the post cross-linking process providing the respective particle with its shell. In such an embodiment, these process parameters or directly the core size and the shell size can depending on the radius also be used as respective input parameters and can be amended additionally or alternatively to the particle size. This allows to take the contributions of the core size and the shell size to the technical application properties into account and provides more possibilities to reach the desired technical application property. For example, in some cases it might not be possible to meet the respective target technical application property based on amending the particle size alone and it might further be necessary to amend also the respective shell size or core size. Thus, the iteration can then also comprise providing in addition to or as alternative to an amended particle size an amended core and/or shell size. Further details on this preferred property determination model will be provided in the following.

Superabsorbent material are commonly produced by grinding and sieving a previously dried gel resulting from a polymerization step. For example, after belt drying the dried gel is initially provided in the form of at least one infinitely long, often several cm thick, flat slab that can then be ground down and sieved to provide a superabsorbent polymer powder as superabsorbent material. For the grinding step, for example, a combination of finger crushers, roller mills or pin mills can be used for grinding down the polymer slab. However, in most common productions processes the grinding step leads to a broad and not necessarily normally distributed particle size distribution of the superabsorbent particles forming the resulting superabsorbent material. Most grinding processes produce both superabsorbent particles that are too fine, for example, smaller than approx. 100 μm, and superabsorbent particles that are too coarse, for example, bigger than approx. 850 μm, for a respective application. The particles that are too fine can often be separated and recycled into the gel or monomer during base superabsorbent polymer production. The too coarse particles can be further comminuted, for example, by repeated grinding or additional grinding stages. However, both process steps are economically disadvantageous and can also undesirably deteriorate the quality of the end product. It is therefore advantageous to limit the further processing steps for the too fine and too coarse particles to the necessary minimum for a respective application.

In recent years for very thin hygiene articles that also may not comprise much fluff, if any, narrower particle sizes are required as described above while they may not contain too many very fine particles for industrial hygiene and performance reasons, e.g. the particle size fraction less than 50 μm, preferably less than 100 μm, most preferably less than 150 μm has to be minimized, and ideally should be absent. Such very fine particles however cannot be avoided in any milling process and must be removed from the final product. Ideally these very fine particles are recycled in the production process but this capability is very limited as they are cross-linked and recycling can be detrimental to the product performance. Hence, it is preferred to control the production process of superabsorbents in a way that these very fine particle fractions are minimized.

The separation of the fine, good and coarse particle fractions is usually done by classification and separation using screening machines utilizing the size difference of the particles, at least partially by air classification utilizing the different air resistance and the different density of the particles, or segregation via a flow behavior of the particles, e.g. in a trickle segregator or by means of a vibratory feeder or other separation processes that take advantage of the different flow behaviors like friction, adhesion, or cohesion of the particles of different sizes. In typical production processes a combination of these separation methods is utilized. A change in the chemical or physical surface properties of the particles, e.g. due to a change in a manufacturing specification, therefore often requires a considerable adjustment of the grinding or classifying process in order to be able to produce a desired particle size distribution in a stable manner. For example, due to a high space-time yield during the production process, a sieve surface can be loaded with significantly more material than in a laboratory scale, which can then result in a significant decrease in the separation efficiency due to the then strongly emphasized flow properties of the particles.

In all these grinding and separation processes, the task is to provide an optimum particle size distribution that causes the superabsorbent material to meet the technical requirements of the customer and also allows to improve the efficiency of the production process, for example, by optimizing the steps used to rework the too small and too coarse particles. For this purpose, it is advantageous to provide suitable process parameter settings for the grinding and separation processes used and to adjust these process parameters settings regularly during continuous production. Currently these process parameter settings and the adjustments are usually provided by an experienced plant operator and can further be based on the analysis of product and/or intermediate samples taken. For this purpose, samples are taken at suitable points in the production process, for example, usually a sample of the base superabsorbent polymer to be fed to the post cross-linking procedure and a sample of the finished product obtained from post cross-linking procedure are analyzed. Furthermore, an attempt is made to record the quantity flows of material during the production process, as far as this is technically possible. The adjustment itself is then often carried out empirically, whereby the problem of optimization arises. The disadvantage is that in practice this is a time-consuming and cost-intensive “trial and error” method. In the case of new equipment in the grinding and separation process or in the case of changes in the base superabsorbent material, e.g. morphology, porosity, surface properties, etc, due to its manufacture, e.g. formulation, polymerization, extrusion, drying, etc., it is often necessary, but often difficult due to a lack of operating experience, especially with new products, to repeatedly work out the necessary settings to allow for a continued product quality, in particular, to allow for the same technical application properties of the final product. Especially for continuous manufacturing processes with high space-time yields, operating trials are often ineffective and difficult to perform. Also, the difference in selectivity between laboratory and production provides additional complexity in scale-up: while the optimum particle size distribution can be developed and adjusted effortlessly in the laboratory, the actual production process usually requires elaborate operational trials and adjustments over a longer period of time. Often, not the same particle size distribution as in the laboratory can be set for the actual production process, but only a best possible approximation.

In production plants, e.g. in crushing or sieving devices, mechanical wear of the equipment also occurs over time, which must be compensated for by regular readjustment of the process parameters so that the set particle size distribution is maintained, e.g. a gap width in roller mills, the selection of the sieves, etc.

The superabsorbent polymer powder produced as describe above is further usually sent to a post cross-linking process step following the generation of an optimum particle size distribution. In this step, a post cross-linking agent dissolved in a solvent is sprayed on the superabsorbent powder, leading to a post cross-linking on the surface of the superabsorbent particles by annealing and dried at least partially or completely. Depending on the formulation composition, the post cross-linking solution penetrates the particles only to a certain depth. Complete penetration of the particles is often not advantageous, since it is usually important to combine the properties of a strongly cross-linked surface shell with an only slightly cross-linked base polymer core. An excessively thin post cross-linking shell is also not advantageous, as this can be damaged by mechanical abrasion, resulting in at least partial loss of the desired particle properties. Generally, experimental determination of shell thickness is known but labor-intensive and only leads to approximated results as described, for example, in “Modern Superabsorbent Polymer Technology”, by F. L. Buchholz, A. T. Graham, Wiley-VCH, Weinheim, 1998, pp. 192-193.

Generally, the above described core-shell structure with the shell polymer covalently bonded to the core polymer and the surface only tearing open during swelling without detaching leads to a strong dependence of almost every technical application property of the end product on the particle size distribution of the superabsorbent polymer powder. During product development and the manufacturing process, usually only the technical application properties of the final produced superabsorbent polymer comprising a broad particle size distribution are determined. In this form, however, the contributions of the superabsorbent polymer powder, surface post cross-linking and particle size distribution are inseparably linked and only appear as an overall performance. Optimization of these three components is therefore typically carried out by means of elaborate tests in the laboratory and in operation, or is not possible with regard to the particle size distribution. This gives rise to the further task of providing possibilities that allow, for example, for an optimization of the cross linking process of the superabsorbent polymer particles together with an optimization of the particle size distribution, wherein a further optimization of post cross-linking process provides more possibilities than the often technically limited optimization of the particle size distribution. Moreover, it could be advantageous if technical application properties of a superabsorbent material could be determined in silico in hygiene articles. Here, it is in particular advantageous to be able to determine the technical application properties of the different superabsorbent particles in respective mixtures usually present, for example, as starting point for a possible product development.

Thus, the invention as described above, for example, with respect to FIG. 1 to 4 provides a possibility to determine a technical application property of superabsorbent particles based on the radius of the superabsorbent particles by utilizing a property determination model. However, in a preferred embodiment that is described in the following in more detail with respect to FIG. 5, it is further preferred that the property determination model also takes the sizes of the core and the shell of the particle into account.

Surprisingly, it was found by the inventors that the contributions of the core and the shell of a superabsorbent particle to a technical application property, which are hardly or not at all accessible experimentally, can be easily separated in a property determination model taking into account the plausible assumption that the sizes, e.g. quantified by the volumes Vₛₕₑₗₗ, V_{core}, of the shell and the core, respectively, in a dry superabsorbent particle are changed in different ways by swelling during use, for instance, different densities of cross-linking lead to different degrees of swelling, but dominate the attainable technical application properties of the swollen particles in use due to their structurally given properties before swelling.

FIG. 5 shows schematically and exemplarily a respective model of swelling of a superabsorbent particle. The particle in the dry state is shown on the left side comprising a core with a radius rᵢ, a shell thickness of d and a particle radius of rₒ such that rₒ=rᵢ+d. At the right side the superabsorbent particle after swelling is shown. As shown during swelling the post cross-linked shell breaks up, but does not separate from the core and retains substantially its size, i.e. volume. Thus, the volumes Vₛₕₑₗₗ, V_{core} of the shell and the core, respectively, can be determined by Vₛₕₑₗₗ=4/3π(ro³−ri³) and V_{core}=4/3πri³, respectively and thus depend on the radius roof the particle and either the shell thickness d or the core radius rᵢ.

To separate the contributions of the shell and the core in the property determination model it is preferably an algorithm used comprising respective performance parameters indicative of the respective contributions that can be learned during a parameterizing of the property determination model. Preferably, a performance parameter varₛₕₑₗₗ quantifies the volume-specific contribution of the shell size, and a performance parameter var_{core} quantifies the volume-specific contribution of the core size. Moreover, since the shell thickness d is often difficult to determine for an actually produced superabsorbent particle, a further performance parameter d′ quantifying the effective thickness of the shell polymer can be utilized to represent d in the model, wherein d′ replaces d for all practical purposes but using the same formulae. Generally, the performance parameter are constant parameters with regard to the particle size but are functions of the formulation and other process parameters, e.g. temperature and residence time in the surface post cross-linking process, etc. Based on these performance parameters a quantity var_{Particle} being or being indicative of the technical application property can be determined. For example, the following relation can be utilized:

$${var}_{Particle} = \frac{{V}_{shell} {var}_{shell} + {V}_{core} {var}_{core}}{{V}_{shell} + {V}_{core}} .$$

Generally, the size, for example, in form of a radius or diameter of the particles is known from a respective size distribution of the superabsorbent particles forming a superabsorbent material. For example, the respective size distribution can be determined by sieving the superabsorbent material or by optical measurements like image analysis, laser diffraction, light barriers, etc., usually with the aid of a calibration versus the sieving method. For example, a Parsum®-probe inline measurement system can be utilized. Such inline measurement is useful because data is readily available for processing and while in the laboratory sieves are highly efficient and reliable classifiers this is not the same in a production plant: throughput and loading on sieve decks will vary and due to wear and tear of the equipment its grinding and classification properties will change. Adjusting and optimizing the process to monitor and take these effects into account in real time greatly benefits from the present invention. In case a performance parameter referring to the thickness of the shell of the superabsorbent particle is utilized, it is noted that this performance parameter quantifies an effective thickness, which may or may not correspond to the physical thickness. For example, the determined value of this performance parameter can vary, for example, in dependency of the technical application property and with the size of the surface of the particles. Moreover, although the above provided exemplary property determination model is discussed with respect to a spherical geometry of the superabsorbent particles, it has been found by the inventors that the above discussed embodiments can also be used to determine the technical application properties of quite irregularly shaped particles, e.g., after extrusion of the gel, very well.

For the above described property determination model the performance parameter can then be determined during a parameterized, in particular, a training, of the property determination model. For example, nonlinear optimization using the “method of least squares” or an equivalently acting optimization function, can be utilized to determine the performance parameter based on historical training data referring to, for instance, laboratory or production samples measurements. In particular, the particles of respective samples comprising a particle size distribution can, for example, be separated in samples comprising only particles with a particles size in a predetermined particle size class, for instance, by sieving the samples with different sieve sizes. The technical application property corresponding to each one of these particle size classes can then be measured for the separate samples, as will be described below. The information on the particle size class and the corresponding technical application property can then be utilized in the training data to determine the performance parameters, which are themselves independent of the size of the particles. For example, during training first initial estimates for the performance parameters are used to calculate the volumes of the shell and core. These calculated volumes can then be substituted into the property determination model utilizing, for example, the above equation, and the technical application property of the particles in that size class can be calculated. Then, the accuracy of the performance parameter and thus the property determination model, can be determined by comparing the calculated technical application property with the measured technical application property of the training data for the respective particle size class. Based on this comparison an optimization function can be utilized to iteratively adjust the performance parameters until the property determination model optimally describes the given measured data. Based on such a training method the property determination model can be parameterized by determining the values for the performance parameters.

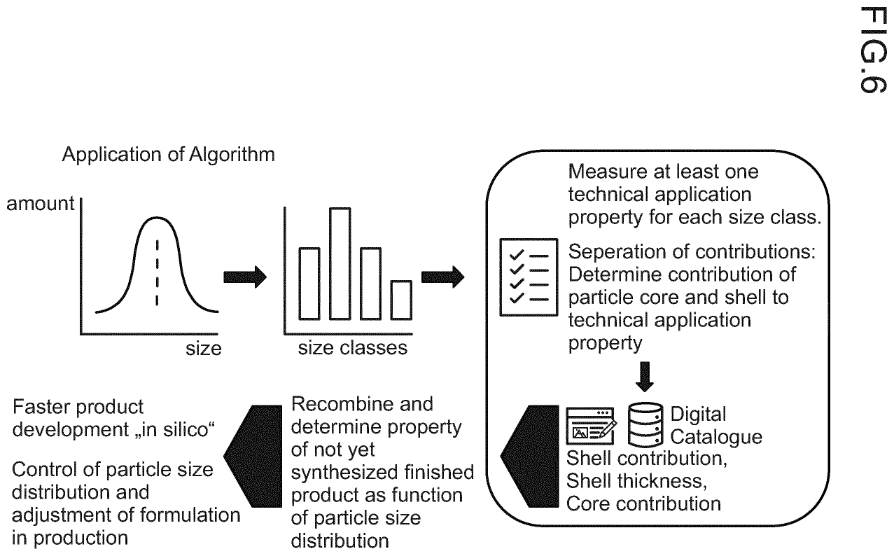

Such a respective training process of a property determination model is shown schematically and exemplarily in FIG. 6. The steps of determining a particle size distribution, and separating a samples of a superabsorbent material into particle size classes is symbolically shown by the first two symbols. Further, the scheme of FIG. 6 includes the determination of a technical application property for each particle size class of the sample. Based on the such obtained training data the performance parameters referring, for example, to the contributions of the core size and the shell size to the technical application property can then be determined, as described above. The performance parameter thus obtained can then be catalogued and stored with respect to formulations and process parameters for which they have been obtained. This storing of the performance parameters equals a storing of the property determination model, since the property determination model is defined by the performance parameters. However, by separating the determined performance parameters for the contribution of the core and surface post cross-linked shell, these performance parameters can also be combined in new ways for providing new property determination models for previously not measured superabsorbent particles. For example, a performance parameter determined for a contribution of a core comprising a superabsorbent polymer can be combined with performance parameters for the contribution of the shell determined for different post cross-linking procedures. In this way a property determination model for a previously not synthesised and measured superabsorbent material can be provided. This makes it possible both to accelerate a product development and to optimize surface post cross-linking procedures, core cross-linking procedures and/or particle size distributions during scale-up in subsequent superabsorbent material production in order to solve the above tasks. In particular, the such determined property determination models can then be used to determine technical application properties of potential superabsorbent materials using the particle size distribution measured, for example, with the above described methods.

In the following some exemplary methods are described that can be utilized for measuring in particularly preferred technical application properties. Generally, EDANA (European Disposables and Nonwovens Association, Avenue Herrmann Debroux 46, 1160 Brussel, Belgien, www.edana.org) and INDA (Association of the Nonwoven Fabrics Industry, 1100 Crescent Green, Suite 115, Cary, North Carolina 27518, U.S.A., www.inda.org) publish joint standard methods “Nonwovens Standards Procedures”, issue 2015, which are available from both organizations, which can be utilized for determining technical application properties in the context of superabsorbent particles. The test methods for superabsorbent materials disclosed in the above publications that determine absorption capacity or permeability are enclosed herein by reference as useful methods for measuring technical application properties in the present invention. Generally, most test methods—if not described otherwise in the method—are executed under an ambient temperature of 23+/−2° C. and a relative humidity of 50+/−10%. Unless indicated otherwise, the granular superabsorbent materials are well-mixed before test method execution. This mixing is particular relevant to obtain a representative sample for the determination of technical application properties as they may vary by particle size. CRC (Centrifuge Retention Capacity) can be determined according to the EDANA Test method NWSP 241.0.R2 (15) “Gravimetric Determination of Fluid Retention Capacity in Saline Solution After Centrifugation”. FSC (Free Swell Capacity in Saline by Gravimetric Determination) can be determined according to EDANA Test method NWSP 240.0.R2 (15). AAP (Absorption against pressure) can be determined according to EDANA Test method Nr. NWSP 242.0.R2 (15) “Absorption Under Pressure, Gravimetric Determination” at one or more pre-determined external pressures depending on the characteristics of the superabsorbent material. External pressures may vary by choice of the applied weight and typical pressures are 0.0, 0.3, 0.7 psi which correspond to 0.0, 21.0, 49.2 g/cm². T20 can be determined as a liquid uptake time for 20 g/g (T20) according to the test procedure described in EP 2 535 027 A1 pages 13-18“K(t) Test Method (Dynamic Effective Permeability and Uptake Kinetics Measurement Test Method)”. VAUL (Volumetric Absorbency Under Load) can be determined by the method described in EP 2 922 882 B1, page 22 and a “characteristic swelling time” usually denoted as Tvalue can also be obtained. The external pressure used in the method may vary between 0.0-0.7 psi, preferred are 0.03 psi or 0.30 psi. Vortex can be determined according to the Vortex Time Method described in F. L. Buchholz, A. T. Graham, Modern Superabsorbent Polymer Technology, Wiley-VCH, Weinheim, 1998, pages 156-157. SFC (Saline Flow Conductivity) can be determined according to EP 2 535 698 A1 pages 19-22 “Urine Permeability Measurement (UPM) Test method”. PSD is the “Standard Test Method for Superabsorbent Materials and the Determination of Polyacrylate Superabsorbent Powders and Particle Size Distribution—Sieve Fractionation” NWSP 220.0.R2 (15). This method is useful to classify the superabsorbent material into predetermined size fractions for determination of technical application properties.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the receiving of the particle size, the determining of the technical application property, the generating of the control data, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or otherwired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include network and/or data links which can be used to carry desired program code means in the form of computer-executable instructions or data structures and which can be accessed by a general-purpose or specialpurpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for determining a technical application property of a superabsorbent material. The superabsorbent material is provided in form of superabsorbent particles comprising a polymer provided in form of i) an interconnected core and ii) a surface cross-linked shell. The apparatus comprises a receiving interface receiving a particle size of the particles of a superabsorbent material. One or more processors are configured to utilize a property determination model for determining the technical application property of the superabsorbent material based on the particle size, wherein the property determination model is a data-driven model that has been parameterized such that it is adapted to determine a technical application property of a superabsorbent particle based on the size of the particle. An output interface generates control data based on the determined technical application property.

1. An apparatus for determining a technical application property of a superabsorbent material, wherein the superabsorbent material is provided in form of superabsorbent particles comprising a superabsorbent polymer provided in form of i) an interconnected core and ii) a surface cross-linked shell with a higher connectivity than the core, wherein the apparatus comprises:
a receiving interface receiving a particle size of the superabsorbent particles of the superabsorbent material,
one or more processors configured to
utilize a property determination model for determining the technical application property of the superabsorbent material based on the particle size, wherein the property determination model is a data-driven model that has been parameterized such that it is adapted to determine a technical application property of a superabsorbent particle based on the size of the particle, and
an output interface for generating control data based on the determined technical application property.

2. The apparatus according to claim 1, wherein the property determination model is further parameterized based on a core size and a shell size of the superabsorbent particle.

3. The apparatus according to claim 2, wherein the parameterizing of utilized property determination model comprises determining performance parameters quantifying a contribution of the core size and the shell size, respectively, of a superabsorbent particle to the technical application property.

4. The apparatus according to claim 1, wherein the utilized property determination model is based on the following relation between the technical application property and a size of a superabsorbent particle:
$${var}_{Particle} = \frac{{V}_{shell} {var}_{shell} + {V}_{core} {var}_{core}}{{V}_{shell} + {V}_{core}}$$
wherein Vₛₕₑₗₗ is a volume of the shell, and V_{core} is a volume of the core of the superabsorbent particle, wherein the volume of the shell and the volume of the core of the superabsorbent particle depend on the size of the superabsorbent particle, and wherein varₛₕₑₗₗ and var_{core} are the performance parameters quantifying the contribution of the core size and the shell size, respectively, and are determined during the parameterization of the property determination model, and wherein var_{Particle} is indicative of the technical application property.

5. The apparatus according to claim 1, wherein the received particle size is a particle size distribution of the superabsorbent particles of the superabsorbent material, and wherein the one or more processors are further configured to a) determine based on the particle size distribution one or more particle size classes from predetermined particle size classes for which particles with respective sizes are present in the superabsorbent material, b) determine a technical application property for the determined particle size classes and c) determine an overall technical application property of the superabsorbent material based on the determined technical application properties for the respective determined particle size classes and based on the particle size distribution.

6. The apparatus according to claim 1, wherein the received particle size is provided as a starting particle size and wherein the receiving interface is further adapted to receive a target application property, wherein the processor is further adapted to iteratively determine a target particle size such that the superabsorbent material meets the target application property within predetermined limits, wherein the iteration comprises a) determining in each iteration step a technical application property, b) comparing the determined technical application property with the target technical application property, and c), based on the comparison, provide an amended particle size, or determine the current particle size as the target size.

7. The apparatus according to claim 1, wherein the generated control data comprises a manufacturing specification for controlling a manufacturing of the superabsorbent material.

8. An interface apparatus for providing an interface for determining a technical application property of a superabsorbent material, wherein the interface apparatus comprises:
an interface input unit capable of interfacing with the apparatus of claim 1 for providing a particle size to that apparatus, and
an interface output unit for processing control data generated by the apparatus according to claim 1 based on the particle size.

9. A training apparatus for parameterizing a property determination model, wherein the training apparatus comprises:
a receiving interface for receiving historical training data comprising a plurality of particle sizes for superabsorbent particles of a superabsorbent material and corresponding one or more measured application properties of the superabsorbent material,
one or more processors configured to utilize the received historical training data for parameterizing a property determination model such that the parameterized property determination model is adapted to determine a technical application property of the superabsorbent material based on a particle size, and
an output interface for outputting the parameterized property determination model.

10. An optimization apparatus for determining a target superabsorbent material comprising a target technical application property, wherein the superabsorbent material is provided in form of superabsorbent particles comprising superabsorbent polymer provided in form of i) an interconnected core and ii) a surface cross-linked shell with a higher connectivity than the core, wherein the apparatus comprises:
a receiving interface receiving the target technical application property for the target superabsorbent material and a particle size of a potential superabsorbent particles of a superabsorbent material,
one or more processors configured to
utilize a property determination model for determining the technical application property of the potential superabsorbent material based on the particle size, wherein the property determination model is a data-driven model that has been parameterized such that it is adapted to determine a technical application property of a superabsorbent particle based on the size of the particle, and
compare the determined technical application property with the target technical application property and determining i) the particle size as the target particle size if the predicted technical application property lies within a predetermined range around the target application property, and ii) an amended particle size and repeating the determination of the technical application property using the amended particle size, if the predicted technical application property lies outside a predetermined range around the target application property, and
an output interface configured for generating a control signal based on the target particle size.

11. (canceled)

12. An interface method for providing an interface for determining a technical application property of a superabsorbent material, wherein the interface method comprises:
providing, via an input interface, a particle size to the apparatus of claim 1, and
processing, via an output interface, control data generated by that apparatus based on the particle size.

13. (canceled)

14. (canceled)

15. A computer program product for determining a technical application property of a superabsorbent material, wherein the computer program product comprises program code means for causing the apparatus of claim 1 to execute a method for determining a technical application property of a superabsorbent material, wherein the superabsorbent material is provided in form of superabsorbent particles comprising i) a core with a superabsorbent polymer and ii) a surface cross-linked shell, wherein the method comprises:
receiving a particle size of the superabsorbent particles of a superabsorbent material,
utilizing a property determination model for determining the technical application property of the superabsorbent material, wherein the property determination model is a data-driven model that has been parameterized such that it is adapted to determine a technical application property of a superabsorbent particle based on the size of the particle, and
generating control data based on the determined technical application property.
